(19) United States
(12) Patent Application Publication
(10) Pub. No.: US 2010/0217100 A1
(43) Pub. Date: Aug. 26, 2010

(54) Methods and Apparatus for Measuring Physiological Conditions

(76) Inventors: Steven Francis LeBoeuf, Raleigh, NC (US); Jesse Berkley Tucker, Knightdale, NC (US); Michael Edward Aumer, Raleigh, NC (US)

Correspondence Address: MYERS BIGEL SIBLEY & SAJOVEC, PO BOX 37428, RALEIGH, NC, 27627, US

(21) Appl. No.: 12/711,736

(22) Filed: Feb. 24, 2010

Related U.S. Application Data
(60) Provisional application No. 61/208,567, filed on Feb. 25, 2009.
(60) Provisional application No. 61/208,574, filed on Feb. 25, 2009.
(60) Provisional application No. 61/212,444, filed on Apr. 13, 2009.
(60) Provisional application No. 61/274,191, filed on Aug. 14, 2009.

(51) Int. Cl. A61B 5/00; A61B 5/04

(52) U.S. Cl. 600/301; 600/382

(57) ABSTRACT

A monitoring apparatus includes a housing configured to be attached to an ear of a subject, and a plurality of electrodes supported by the housing. The electrodes are configured to at least partially contact a portion of the body of the subject when the housing is attached to the ear of the subject, and are configured to detect and/or measure at least one neurological and/or cardiopulmonary function of the subject. The housing may include one or more physiological sensors configured to detect and/or measure physiological information from the subject and/or one or more environmental sensors configured to detect and/or measure environmental conditions in a vicinity of the subject.

### RELATED APPLICATIONS

This application claims the benefit of and priority to U.S. Provisional Patent Application No. 61/208,567 filed Feb. 25, 2009, U.S. Provisional Patent Application No. 61/208,574 filed Feb. 25, 2009, U.S. Provisional Patent Application No. 61/212,444 filed Apr. 13, 2009, and U.S. Provisional Patent Application No. 61/274,191 filed Aug. 14, 2009, the disclosures of which are incorporated herein by reference as if set forth in their entireties.

### FIELD OF THE INVENTION

The present invention relates generally to health and, more particularly, to health monitoring methods and apparatus.

### BACKGROUND OF THE INVENTION

There is growing market demand for personal health monitors, for example, for gauging overall health and metabolism of persons during exercise, athletic training, dieting, and physical therapy. Various physiological information, such as electrocardiogram (ECG) information, electroencephalogram (EEG) information, electrooculography (EOG) information, and other forms of physiological electrical activity, may be useful to monitor during physical activity. However, traditional monitors for measuring this type of information may be bulky, rigid, non-portable, and uncomfortable—generally not suitable for use during physical activity.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Embodiments of the present invention provide novel devices and methods for noninvasively qualifying and/or quantifying physiological information from a subject, such as neurological and cardio-pulmonary information, with various electrodes embedded in an audio headset. According to some embodiments of the present invention, a monitoring apparatus includes a housing configured to be attached to an ear of a subject, and a plurality of electrodes supported by the housing. The electrodes are configured to at least partially contact a portion of the body of the subject when the housing is attached to the ear of the subject, and are configured to detect and/or measure at least one neurological and/or cardiopulmonary function of the subject. Exemplary electrodes that may be utilized include, but are not limited to, electrocardiogram (ECG) electrodes, electroencephalogram (EEG) electrodes, and electrooculography (EOG) electrodes. To ensure good contact with the skin of a subject, the housing may include one or more biasing members or other structures that urge the electrodes into contact with the body of the subject when the housing is attached to the ear of the subject. In addition to electrodes, monitoring apparatus, according to some embodiments of the present invention, may include one or more physiological sensors configured to detect and/or measure physiological information from the subject and/or one or more environmental sensors configured to detect and/or measure environmental conditions in a vicinity of the subject.

In some embodiments of the present invention, a sensor module is included with circuitry that is configured to amplify and/or filter signals produced by the electrodes. In some embodiments, the circuitry comprises a microcontroller. In some embodiments, the sensor module is configured to digitize signals produced by the electrodes. The monitoring apparatus may include a power conditioning component configured to adjust voltage and/or current to the sensor module. A transmitter may be included that is configured to transmit signals processed by the sensor module to a remote device.

In some embodiments, the monitoring apparatus includes a speaker and microphone supported by the housing. The speaker is in electrical communication with an electronic device via an audio output port of the electronic device, and the microphone is in electrical communication with the electronic device via an audio input port of the electronic device. The sensor module modulates and transmits signals produced by the electrodes to the electronic device via the audio input port. In other embodiments, however, the sensor module may be configured to wirelessly transmit signals produced by the electrodes to a remote electronic device.

In some embodiments, circuitry and sensor electrodes are integrated into a sensor control module that processes sensor signals and transmits these signals to another device. In a specific case, the circuitry may comprise a microcontroller, transmitter, sensor electrodes, and additional sensor circuitry.

In some embodiments, the monitoring apparatus is a headset having an ear clip that facilitates attachment of the housing to the ear of a subject. The ear clip may include one or more electrodes configured to at least partially contact a portion of the subject's body when the housing is attached to the ear. In some embodiments, the ear clip may include a pinna cover having one or more electrodes configured to at least partially contact a portion of the ear.

In some embodiments, the monitoring apparatus is an earbud configured to be inserted within an ear of a subject. The earbud includes electrodes configured to at least partially contact a portion of the ear of the subject when the earbud is inserted within the ear of the subject.

In some embodiments, the headset includes two earbuds connected by a supporting member, wherein each earbud is configured to be inserted within a respective ear of a subject. The electrodes may be supported by the supporting member and/or one or both of the earbuds.

According to other embodiments of the present invention, a monitoring apparatus includes a headset configured to be worn by a subject and an electronic device having a user interface. The electronic device may be worn by the subject (e.g., on the body of the subject and/or attached to clothing, etc.). The headset includes a plurality of electrodes (e.g., ECG electrodes, EEG electrodes, EOG electrodes) configured to at least partially contact a portion of the body of the subject when the headset is worn by the subject. The electrodes are configured to detect and/or measure at least one neurological and/or cardiopulmonary function of the subject. The headset also includes a sensor module configured to receive and transmit signals produced by the electrodes to the electronic device for display via the user interface of the electronic device. The sensor module may also be configured to amplify and/or filter signals produced by the electrodes

To ensure good contact with the skin of a subject, the headset may include one or more biasing members or other structures that urge the electrodes into contact with the body of the subject when the headset is attached to the ear of the subject. In addition to electrodes, the headset may include one or more physiological sensors configured to detect and/or measure physiological information from the subject and/or one or more environmental sensors configured to detect and/or measure environmental conditions in a vicinity of the subject.

In some embodiments, the headset includes a speaker and a microphone. The speaker is in electrical communication with the electronic device via an audio output port of the electronic device, and the microphone is in electrical communication with the electronic device via an audio input port of the electronic device. The sensor module is configured to modulate and transmit signals produced by the electrodes to the electronic device via the audio input port.

According to other embodiments of the present invention, a monitoring apparatus includes a housing configured to be attached to an ear of a subject, a first electrode supported by the housing and configured to at least partially contact a portion of the body of the subject when the housing is attached to the ear of the subject, an earring configured to be attached to the ear of the subject, and a second electrode supported by the earring and configured to at least partially contact a portion of the ear of the subject when the earring is attached to the ear of the subject. The first and second electrodes are configured to detect and/or measure at least one neurological and/or cardiopulmonary function of the subject. Exemplary electrodes include ECG electrodes, EEG electrodes, EOG electrodes. In some embodiments, the monitoring apparatus includes a sensor module supported by the housing and configured to amplify and/or filter signals produced by the first and second electrodes. In some embodiments, the monitoring apparatus includes a transmitter supported by the housing and configured to transmit signals processed by the sensor module to a remote device.

According to other embodiments of the present invention, a method of monitoring a subject includes detecting neurological and/or cardiopulmonary function information from the subject via electrodes (e.g., ECG electrodes, EEG electrodes, EOG electrodes, etc.) attached to a headset worn by the subject, and transmitting the information to a remote electronic device via an audio input port of the remote electronic device. In some embodiments the headset includes a microphone in electrical communication with the electronic device via an audio input port of the electronic device. Transmitting information to the remote electronic device includes modulating the information and transmitting the information with audio signals produced by the microphone. In some embodiments, transmitting information to the remote electronic device is performed wirelessly.

Because headsets have been adopted for widespread everyday use, embodiments of the present invention provide a convenient and unobtrusive way of monitoring various neurological and cardio-pulmonary functions. Moreover, because the ear region is located next to a variety of “hot spots” for physiological an environmental sensing, including the tympanic membrane, the carotid artery, the paranasal sinus, etc., headsets, according to embodiments of the present invention, are advantageous over other types of monitoring devices configured for other parts of the body. In addition, monitoring apparatus according to embodiments of the present invention can leverage both the bilateral symmetry and asymmetry of the human body. For example, a potential can be measured across the left and right side of the body during the electrical generation of a systolic heart event. For this reason, a net potential may be measured from ear-to-ear during the generation of a heartbeat.

Monitoring apparatus, according to embodiments of the present invention, can utilize commercially available open-architecture, ad hoc, wireless paradigms, such as Bluetooth®, Wi-Fi, or ZigBee. In some embodiments, a small, compact earpiece contains at least one microphone and one speaker, and is configured to transmit information wirelessly to a recording device such as, for example, a cell phone, a personal digital assistant (PDA), and/or a computer. The earpiece contains a plurality of sensors for monitoring personal health and environmental exposure. Health and environmental information, sensed by the sensors can be transmitted wirelessly, in real-time, to a recording device, capable of processing and organizing the data into meaningful displays, such as charts. In some embodiments, an earpiece user can monitor health and environmental exposure data in real-time, and may also access records of collected data throughout the day, week, month, etc., by observing charts and data through an audio-visual display.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 illustrates a monitoring apparatus, according to some embodiments of the present invention, that includes a headset and a remote electronic device.

FIG. 2 illustrates the remote electronic device of FIG. 1 attached to the arm of a subject.

FIG. 3 illustrates a circuit for extracting an ECG signal from the ear of a subject.

FIG. 4 illustrates the anatomy of a human ear.

FIG. 5 illustrates a monitoring apparatus in the form of an earbud, according to some embodiments of the present invention, near the ear of a subject.

FIG. 6 is a perspective view of a headset with embedded electrodes, according to some embodiments of the present invention.

FIG. 7 is a perspective view of a headset monitoring apparatus, according to some embodiments of the present invention.

FIG. 8 illustrates a flexible electrode/sensor module that may be utilized within monitoring apparatus according to some embodiments of the present invention.

FIG. 9 illustrates a monitoring apparatus in the form of a headset and earring, according to some embodiments of the present invention.

FIGS. 10-12 are block diagrams of monitoring apparatus, according to some embodiments of the present invention.

### DETAILED DESCRIPTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that although the terms first and second are used herein to describe various features/elements, these features/elements should not be limited by these terms. These terms are only used to distinguish one feature/element from another feature/element. Thus, a first feature/element discussed below could be termed a second feature/element, and similarly, a second feature/element discussed below could be termed a first feature/element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “headset” includes any type of device or earpiece that may be attached to or near the ear (or ears) of a user and may have various configurations, without limitation. Headsets, as described herein, may include mono headsets (one earbud) and stereo headsets (two earbuds). The term “earpiece module” includes any type of device that may be attached to or near the ear of a user and may have various configurations, without limitation. The terms “headset” and “earpiece module” may be interchangeable.

The term “real-time” is used to describe a process of sensing, processing, or transmitting information in a time frame which is equal to or shorter than the minimum timescale at which the information is needed. For example, the real-time monitoring of pulse rate may result in a single average pulse-rate measurement every minute, averaged over 30 seconds, because an instantaneous pulse rate is often useless to the end user. Typically, averaged physiological and environmental information is more relevant than instantaneous changes. Thus, in the context of the present invention, signals may sometimes be processed over several seconds, or even minutes, in order to generate a “real-time” response.

The term “monitoring” refers to the act of measuring, quantifying, qualifying, estimating, sensing, calculating, interpolating, extrapolating, inferring, deducing, or any combination of these actions. More generally, “monitoring” refers to a way of getting information via one or more sensing elements (e.g., physiological sensors, environmental sensors, etc.). For example, “blood health monitoring” includes monitoring blood gas levels, blood hydration, and metabolite/electrolyte levels.

The term “physiological” refers to matter or energy of or from the body of a subject (e.g., humans, animals, etc.). In embodiments of the present invention, the term “physiological” is intended to be used broadly, covering both physical and psychological matter and energy of or from the body of a creature. However, in some cases, the term “psychological” is called-out separately to emphasize aspects of physiology that are more closely tied to conscious or subconscious brain activity rather than the activity of other organs, tissues, or cells.

The term “environmental exposure” refers to any environmental occurrence (or energy) to which an individual or group of individuals is exposed. For example, exposure to solar energy, air pollution, temperature, nuclear radiation, humidity, water, etc. may all constitute environmental exposure. A variety of relevant environmental energies are listed elsewhere herein.

The term “body” refers to the body of a subject (human or animal) that may wear a monitoring apparatus, according to embodiments of the present invention.

The term “health” refers generally to the quality or quantity of one or more physiological parameters with reference to an subject's functional abilities.

The term “processor” refers to a device that takes one form of information and converts this information into another form, typically having more usefulness than the original form. For example, in this invention, a signal processor may collect raw physiological or environmental data from various sensors and process this data into a meaningful assessment, such as pulse rate, blood pressure, or air quality. A variety of microprocessors or other processors may be used herein. The terms “signal processor”, “processor”, “controller”, and “microcontroller”, as used herein, are interchangeable.

Some embodiments of the present invention arise from a discovery that the ear is an ideal location on the human body for a wearable health and environmental monitor. The ear is a relatively immobile platform that does not obstruct a person's movement or vision. Devices located along the ear can have access to the inner-ear canal and tympanic membrane (for measuring core body temperature), muscle tissue (for monitoring muscle tension), the pinna and earlobe (for monitoring blood gas levels), the region behind the ear (for measuring skin temperature and galvanic skin response), and the internal carotid artery (for measuring cardiopulmonary functioning). The ear is also at or near the point of exposure to: environmental breathable toxicants of interest (volatile organic compounds, pollution, etc.); noise pollution experienced by the ear; and lighting conditions for the eye. Located adjacent to the brain, the ear serves as an excellent location for mounting neurological and electrical electrodes/sensors for monitoring brain activity. Furthermore, as the ear canal is naturally designed for transmitting acoustical energy, the ear provides an optimal location for monitoring internal sounds, such as heartbeat, breathing rate, and mouth motion.

In the following figures, headsets, earpiece modules, and other monitoring apparatus will be illustrated and described for attachment to or near the ear of the human body. However, it is to be understood that embodiments of the present invention are not limited to those worn by humans.

According to some embodiments of the present invention, monitoring apparatus for attachment to or near the ear of a subject include various types of headsets, including wired or wireless headsets. Wired or wireless headsets, such as Bluetooth®-enabled and/or other personal communication headsets, may be configured to incorporate electrodes and physiological and/or environmental sensors, according to some embodiments of the present invention. Bluetooth® headsets are typically lightweight, unobtrusive devices that have become widely accepted socially. Moreover, Bluetooth® headsets may be cost effective, easy to use, and are often worn by users for most of their waking hours while attending or waiting for cell phone calls. Bluetooth® headsets configured according to embodiments of the present invention are advantageous because they provide a function for the user beyond health monitoring, such as personal communication and multimedia applications, thereby encouraging user compliance with monitoring. Exemplary physiological and environmental sensors that may be incorporated into a Bluetooth® or other type of headset include, but are not limited to accelerometers, auscultatory sensors, pressure sensors, humidity sensors, color sensors, light intensity sensors, pressure sensors, noise signal detectors, etc.

Headsets, both mono (single earbud) and stereo (dual earbuds), incorporating low-profile electrodes, sensors and other electronics, according to embodiments of the present invention, offer a platform for performing near-real-time personal health and environmental monitoring in wearable, socially acceptable devices. The capability to unobtrusively monitor an individual's physiology and/or environment, combined with improved user compliance, is expected to have significant impact on future planned health and environmental exposure studies. This is especially true for those that seek to link environmental stressors with personal stress level indicators. The large scale commercial availability of low-cost headset devices can enable cost-effective large scale studies. The combination of monitored data with user location via GPS data can make on-going geographic studies possible, including the tracking of infection over large geographic areas. The commercial application of the various proposed platforms encourages individual-driven health maintenance and promotes a healthier lifestyle through proper caloric intake and exercise.

Accordingly, some embodiments of the present invention combine a personal communications and/or entertainment headset device with one or more electrodes and/or one or more physiological and/or environmental sensors. Embodiments of the present invention are not limited to headsets that communicate wirelessly. In some embodiments of the present invention, headsets configured to monitor an individual's physiology and/or environment may be wired to a device that stores and/or processes data. In some embodiments, this information may be stored on the headset itself.

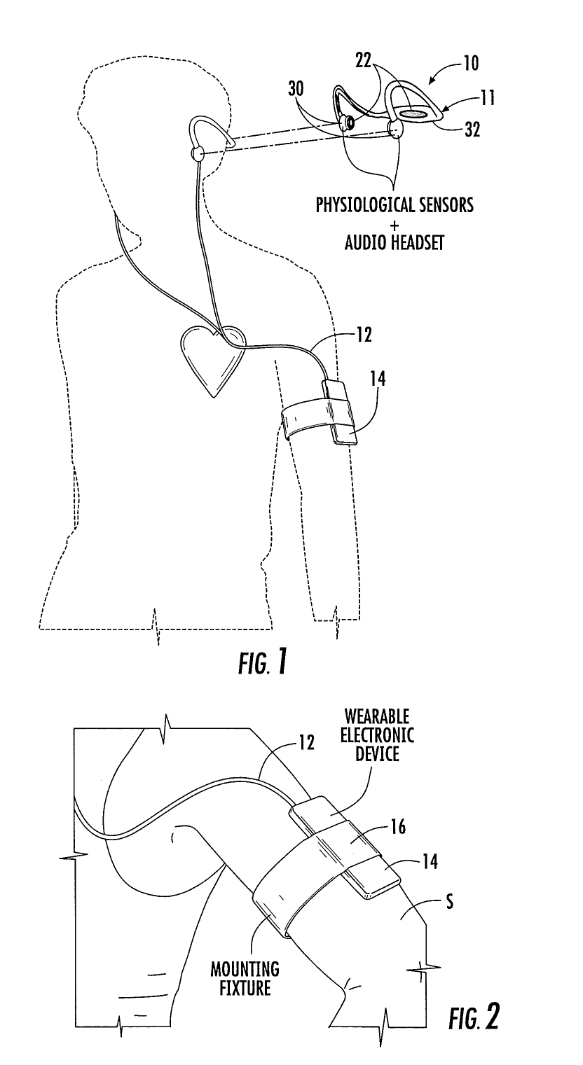

FIG. 1 illustrates a novel, non-limiting embodiment of a monitoring apparatus 10 for monitoring the physiological properties of a subject. More specifically, the illustrated monitoring apparatus 10 includes a headset 11 which integrates electrodes 22 (FIG. 3) and/or sensors (not shown) for monitoring one or more neurological and/or cardio-pulmonary functions of a subject. The headset 11 can be designed to function as both an audio headset and a physiological monitor while maintaining essentially the same form-factor of an audio headset. The electrodes 22 are configured to at least partially contact a portion of the body of the subject when the headset 11 is attached to the subject. Exemplary electrodes that may be utilized include, but are not limited to, electrocardiogram (ECG) electrodes, electroencephalogram (EEG) electrodes, and electrooculography (EOG) electrodes. To ensure good contact with the skin of a subject, the headset 11 may include one or more biasing members or other structures (not shown) that are configured to urge the electrodes into contact with the body of the subject when the headset 11 is attached to the subject.

In addition to electrodes, the headset 11 may include one or more physiological sensors configured to detect and/or measure physiological information from a subject and/or one or more environmental sensors configured to detect and/or measure environmental conditions in a vicinity of a subject. A physiological sensor can be any compact sensor for monitoring the physiological functioning of the body, such as, but not limited to, sensors for monitoring: heart rate, pulse rate, breathing rate, blood flow, heartbeat signatures, cardio-pulmonary health, organ health, metabolism, electrolyte type and concentration, physical activity, caloric intake, caloric metabolism, metabolomics, physical and psychological stress levels and stress level indicators, physiological and psychological response to therapy, drug dosage and activity (drug dosimetry), physiological drug reactions, drug chemistry in the body, biochemistry, position & balance, body strain, neurological functioning, brain activity, brain waves, blood pressure, cranial pressure, hydration level, auscultatory information, auscultatory signals associated with pregnancy, physiological response to infection, skin and core body temperature, eye muscle movement, blood volume, inhaled and exhaled breath volume, physical exertion, exhaled breath physical and chemical composition, the presence, identity, and concentration of viruses & bacteria, foreign matter in the body, internal toxins, heavy metals in the body, anxiety, fertility, ovulation, sex hormones, psychological mood, sleep patterns, hunger & thirst, hormone type and concentration, cholesterol, lipids, blood panel, bone density, body fat density, muscle density, organ and body weight, reflex response, sexual arousal, mental and physical alertness, sleepiness, auscultatory information, response to external stimuli, swallowing volume, swallowing rate, sickness, voice characteristics, tone, pitch, and volume of the voice, vital signs, head tilt, allergic reactions, inflammation response, autoimmune response, mutagenic response, DNA, proteins, protein levels in the blood, body hydration, water content of the blood, pheromones, internal body sounds, digestive system functioning, cellular regeneration response, healing response, stem cell regeneration response, and the like. Vital signs can include pulse rate, breathing rate, blood pressure, pulse signature, body temperature, hydration level, skin temperature, and the like. A physiological sensor may include an impedance plethysmograph for measuring changes in volume within an organ or body (usually resulting from fluctuations in the amount of blood or air it contains). For example, the wearable monitoring device 10 may include an impedance plethysmograph to monitor blood pressure in real-time.

An external energy sensor, serving primarily as an environmental sensor, can be any compact sensor for monitoring the external environment in the vicinity of the body, such as, but not limited to, sensors for monitoring: climate, humidity, temperature, pressure, barometric pressure, pollution, automobile exhaust, soot density, airborne particle density, airborne particle size, airborne particle shape, airborne particle identity, volatile organic chemicals (VOCs), hydrocarbons, polycyclic aromatic hydrocarbons (PAHs), carcinogens, toxins, electromagnetic energy (optical radiation, X-rays, gamma rays, microwave radiation, terahertz radiation, ultraviolet radiation, infrared radiation, radio waves, and the like), EMF energy, atomic energy (alpha particles, beta-particles, gamma rays, and the like), gravity, light properties (such as intensity, frequency, flicker, and phase), ozone, carbon monoxide, greenhouse gases, CO2, nitrous oxide, sulfides, airborne pollution, foreign material in the air, biological particles (viruses, bacteria, and toxins), signatures from chemical weapons, wind, air turbulence, sound and acoustical energy (both human audible and inaudible), ultrasonic energy, noise pollution, human voices, animal sounds, diseases expelled from others, the exhaled breath and breath constituents of others, toxins from others, bacteria & viruses from others, pheromones from others, industrial and transportation sounds, allergens, animal hair, pollen, exhaust from engines, vapors & fumes, fuel, signatures for mineral deposits or oil deposits, snow, rain, thermal energy, hot surfaces, hot gases, solar energy, hail, ice, vibrations, traffic, the number of people in a vicinity of the user, the number of people encountered throughout the day, other earpiece module users in the vicinity of the earpiece module user, coughing and sneezing sounds from people in the vicinity of the user, loudness and pitch from those speaking in the vicinity of the user, and the like.

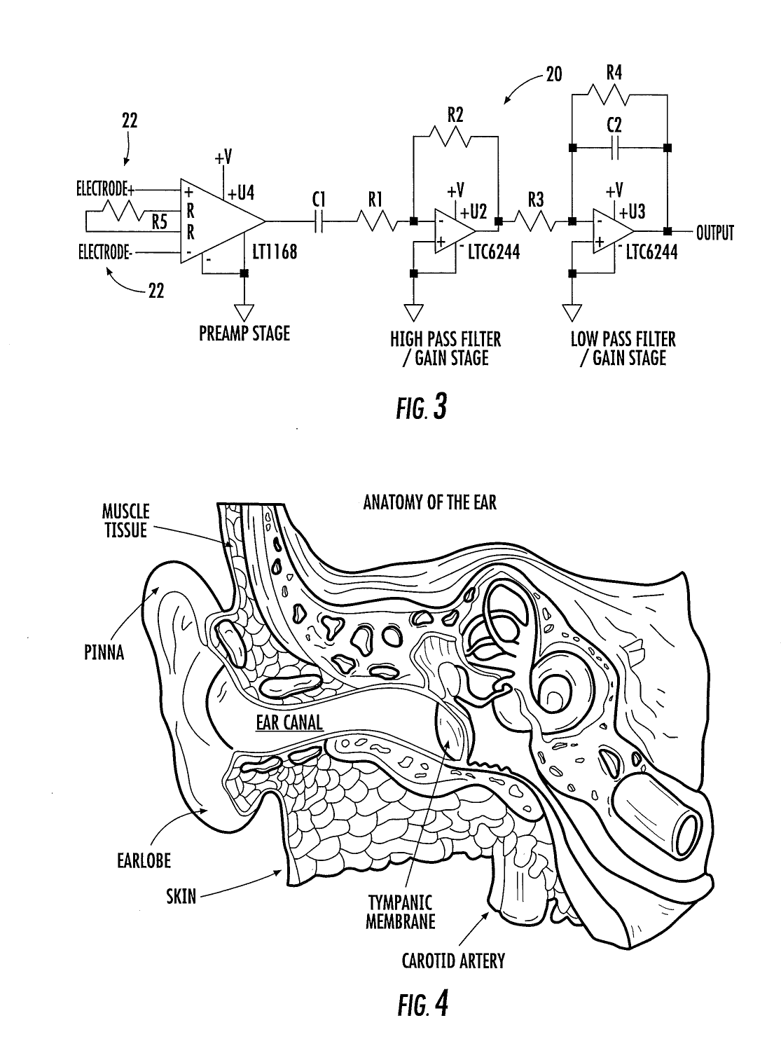

As shown in FIG. 1, the headset 11 may connect via a wire 12 to a wearable electronic device 14, though wireless designs are also possible. The wearable electronic device 14 can be any of a variety of wearable devices including, but not limited to, a cellular phone, a smartphone, a digital media player, Walkman®, a personal digital assistant (PDA), a watch, electronic armband, medallion, or the like. In some embodiments, the wearable electronic device can display, audibly, visually, or both, raw or processed information received by the headset 11 via a user interface. The wearable electronic device 14 may be an embedded system or embedded computer. FIG. 2 shows an example of the wearable electronic device 14 worn on the arm of a subject. In the illustrated embodiment, the electronic device 14 is affixed to an arm support 16, such as an armband.

FIG. 3 shows an exemplary, nonlimiting electronic circuit 20 for extracting ECG signals from the ear region via electrodes 22 and generating an output. In the illustrated embodiment, multiple gain stages are used to generate a bandpass filter centered in the prime region of an ECG response. Typically, this region will range from 40 Hz to 200 Hz.

FIG. 4 shows a summary of the anatomy of the human ear, where there are several locations suitable for contact with electrodes, such as ECG electrodes. Optimal places include regions where there is a reasonably conductive skin area, such as a region with sweat pores. Nonlimiting skin contact locations for electrodes include: the ear canal, the meatus, the pinna, the scapha, the helix, the tragus, the earlobe, and the periphery surrounding the region where the ear meets the head.

Electrodes 22 utilized in monitoring apparatus, according to embodiments of the present invention, may be composed of any conductive material or materials that are solid or gel-like, including, but not limited to: metals, conductive polymers, conductive gels or sol-gels, alloys, conductive plastics/rubbers, semimetals or semiconductors, and the like. Silver/silver chloride electrodes, carbon rubber, copper, and gold electrodes are just a few examples of electrode materials. Electrodes, according to embodiments of the present invention, need not be passive electrodes. In fact, active electrodes can be employed for impedance matching, impedance reduction, and noise reduction. Active electrodes may employ operational amplifiers, voltage followers, impedance-cancelling circuits, or the like. Furthermore, some electrodes may be configured to measure mostly motion noise, and provide a suitable noise reference for removing noise from an ECG signal. In such case, the noise-detection electrodes may be located in regions without a significant ECG potential drop, such that changes in motion generate a higher potential signal than internal ECG signals from the body. Alternatively, the noise-detection electrodes may be designed to have high impedance to the human body to prevent the pickup of ECG signals, picking up mostly motion-related noise.

Electrodes 22, according to embodiments of the present invention, can be located along any part of a headset touching the skin. Preferably, the electrodes are located in a headset region that is always in contact with the skin during use. Compression fixtures, such as biasing members (e.g., springs, etc.) or other structures, can be used to press the electrodes more closely against the skin. Gels, conductive gels, liquids, lubricants, or the like can be applied to the electrodes to improve the signal-to-noise ratio of signals, such as electrocardiograms, measured. In the illustrated embodiment, the headset 11 includes two earbuds 30 connected by a supporting member 32. Each earbud 30 is configured to be inserted within an ear of a subject. One or more electrodes 22 are supported by the supporting member 32, and one or more electrodes 22 are supported by each earbud 30. In other embodiments, electrodes may be located in only one earbud. In some embodiment, the supporting member 32 may not include electrodes.

In the illustrated embodiment, the supporting member 32 may include one or more biasing members (e.g., a spring) or other structures (not shown) that are configured to urge an electrode 22 into contact with the body of the subject when the headset 11 is worn by the subject. In some embodiments, the supporting member 32 may also help compress the electrodes 22 against the skin to maintain electrode contact. In addition, each earbud 30 having electrodes therein may also include one or more biasing members or other structures that are configured to urge an electrode 22 into contact with the ear of the subject.

In some embodiments, additional electrodes may be integrated with the headset electrodes for a more complete heart monitoring platform. For example, at least one electrode near the leg or ankle may serve as a good ground reference. These additional electrodes may be directly connected to the headset 11 via a wire or may be wirelessly connected to the headset 11.

In another embodiment, at least one electrode 22 may be integrated within the wearable electronic device 14, as this device may be worn in such as way that it is always in contact with human skin S (FIG. 2). In other embodiments, chest electrodes may be integrated within the circuit for assessed multiple chambers and functions of the heart. In each case, the “hub” for collecting, powering, and/or processing this data may be within the headset 11 itself or the wearable electronic device 14. For example, all electrodes 22 may complete a circuit within the wearable electronic device 14 or headset 11.

Referring to FIG. 5, one or more electrodes 22 are located on the outer periphery 31 of the illustrated earbud 30, such that the electrodes 22 are in direct contact with the skin of the mid-to-inner ear region when the earbud 30 is inserted within an ear. The electrodes 22 extend circumferentially around the audio passageway 33 in the illustrated earbud 30. However, in other embodiments, the electrodes 22 may extend circumferentially around only a portion of the audio passageway 33. In some embodiments, a single electrode 22 may be located on an earbud 30. However, in other embodiments, multiple electrodes 22 may be located on an earbud 30. Moreover, multiple electrodes 22 of various shapes and orientations can be located on a single earbud 30.

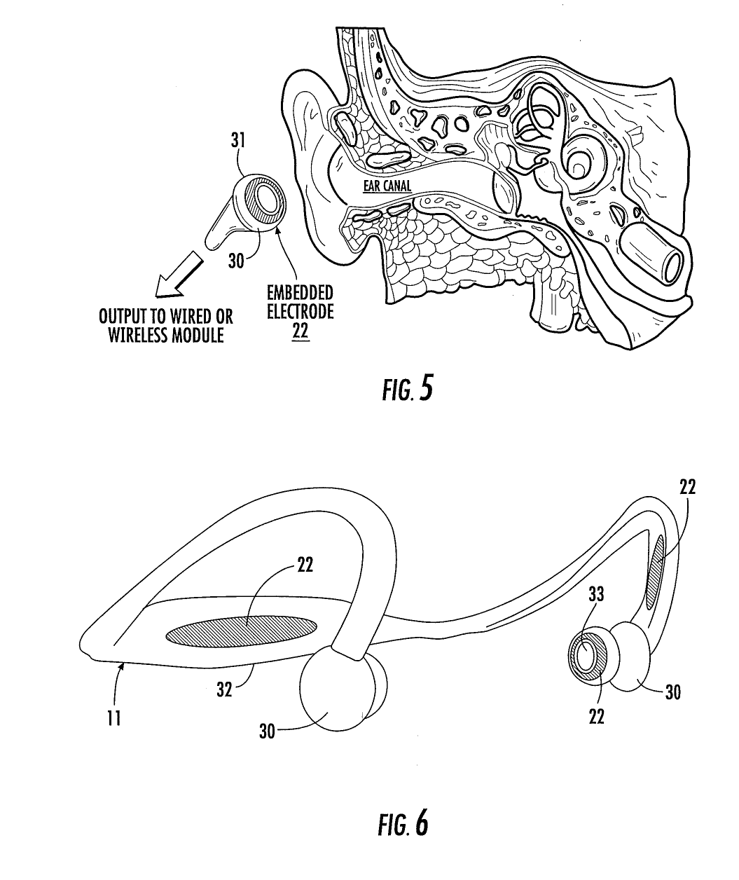

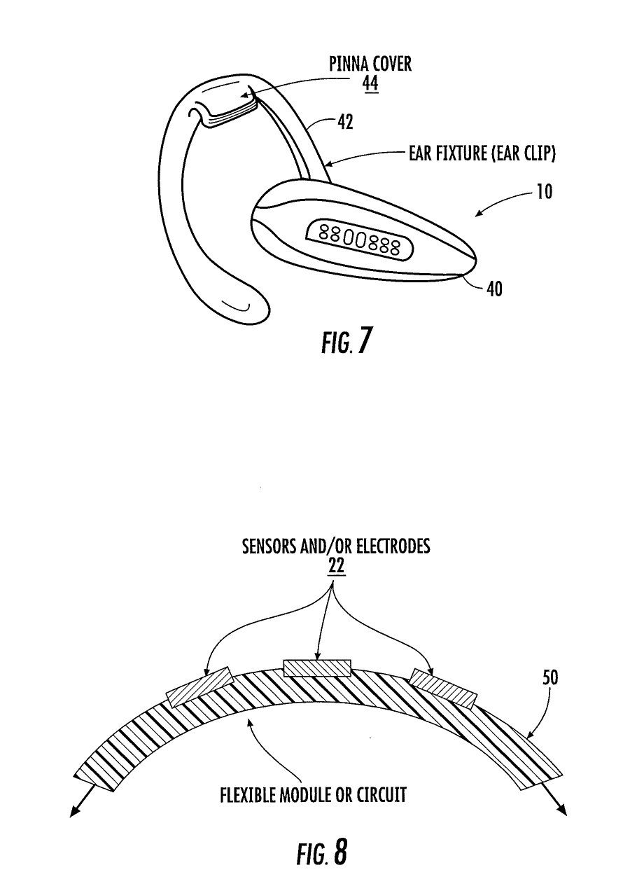

FIG. 6 is an enlarged view of the headset 11 of FIG. 1 and illustrated electrodes 22 embedded into various locations of the headset 11. In the illustrated embodiment, electrodes 22 are shown embedded in the earbud 30, the ear fixture 34, and a back-of-head supporting member 32. Having more than two electrodes in the headset 11 provides a method of extracting cleaner signals, such as ECG signals, from noise.

Referring to FIG. 7, a monitoring apparatus 10, according to other embodiments of the present invention, is illustrated. The illustrated monitoring apparatus 10 includes a housing 40 configured to be attached to an ear of a subject. The illustrated monitoring apparatus 10 also includes an ear clip 42 attached to the housing 40 and that is configured to facilitate attachment of the housing 40 to the ear of a subject. The monitoring apparatus 40 includes a plurality of electrodes (not shown) supported by the housing, and that are configured to at least partially contact a portion of the body of the subject when the housing 40 is attached to the ear of the subject. The electrodes are configured to detect and/or measure at least one neurological and/or cardiopulmonary function of the subject, and may include, for example, ECG electrodes, EEG electrodes, and/or EOG electrodes. In some embodiments, the ear clip 42 may include one or more electrodes configured to at least partially contact a portion of the body of a subject when the housing 40 is attached to the ear of the subject. For example, electrodes may be located in the back (skin-facing) side of the ear clip 42.

In the illustrated embodiment, the ear clip 42 includes a pinna cover 44. The pinna cover 44 may include one or more electrodes configured to at least partially contact a portion of an ear of a subject when the housing 40 is attached to the ear of the subject.

In some embodiments of the present invention, electrodes 22 may be integrated into flexible modules for a snugger, more comfortable, and/or more reliable electrode configuration. FIG. 8 shows an example of a flexible circuit board 50, according to embodiments of the present invention, that can be made out of virtually any stable flexible material, such as kapton, polymers, flexible ceramics, flexible glasses, rubber, and the like. The flexible material of the flexible circuit board is sufficiently electrically insulating and/or electrochemically inert in comparison with electrodes 22 attached thereto. As with a standard rigid circuit board, a variety of electrodes 22 and/or sensors can be mounted on the flexible circuit board 50, and this board 50 can be integrated into any part of a monitoring apparatus 10. Flexible circuitry can be especially useful for odd-shaped components of an earpiece. In some cases, flexible piezoelectric polymers, such as polyvinylidene fluoride may be useful for measuring body motion, arterial motion, and auscultatory sounds from the body.

Ear jewelry, such as an ear piercing or clip-on jewelry, can also be used to help measure neurological and/or cardio-pulmonary functions from a subject, according to some embodiments of the present invention. In such case, electrode wires can be attached to at least one piercing (such as an earring) on each ear of a user, such that the piercing serves as an electrode. Earrings and similar structures may be particularly effective at measuring ECG (and other) signals because they may be highly fixed, localized, and in intimate contact with the skin.

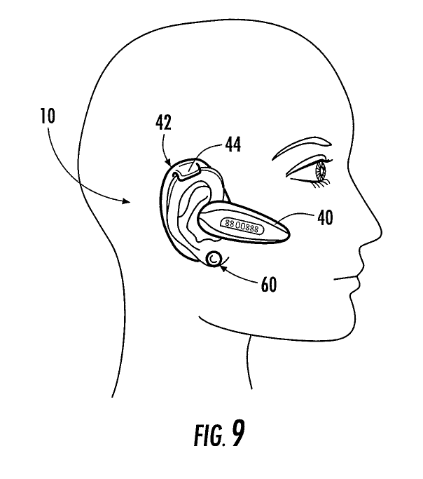

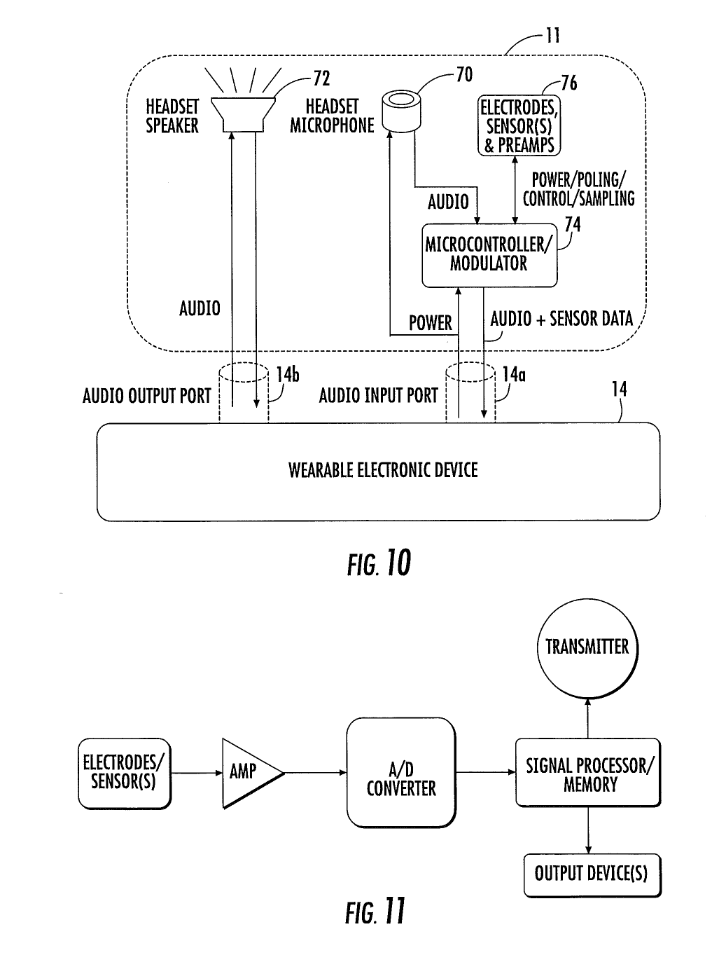

FIG. 9 illustrates a monitoring apparatus 10 that utilizes an earring, according to some embodiments of the present invention. The illustrated monitoring apparatus 10 includes a housing 40 and an earring 60 configured to be attached to an ear of a subject. The housing 40 includes one or more electrodes configured to at least partially contact a portion of the body of the subject when the housing is attached to the ear of the subject. The earring 60 includes one or more electrodes configured to at least partially contact a portion of the ear of the subject when the earring is attached to the ear of the subject. The electrodes supported by the housing 40 and earring 60 are configured to detect and/or measure at least one neurological and/or cardiopulmonary function of the subject. Exemplary electrodes include ECG electrodes, EEG electrodes, EOG electrodes. In some embodiments, the monitoring apparatus 10 includes a sensor module supported by the housing 40 and configured to amplify and/or filter signals produced by the electrodes. In some embodiments, the monitoring apparatus includes a transmitter supported by the housing 40 and configured to transmit signals processed by the sensor module to a remote device.

In the illustrated embodiment of FIG. 9, an ear clip 42 is attached to the housing 40 and includes a pinna cover 44. However, embodiments of the present invention are not limited to the illustrated monitoring apparatus 10. An earring 60 having one or more electrodes may be utilized with various types of headsets, earbuds, etc., without limitation.

Referring to FIG. 10, a monitoring apparatus 10 includes an electronic device 14 and headset 11, such as an earbud module, connected to the electronic device 14, and having a plurality of electrodes configured to at least partially contact a portion of the body of a subject when the headset 10 is worn by the subject. The headset 11 includes a plurality of electrodes configured to at least partially contact a portion of the body of the subject when the headset 11 is worn by the subject and configured to detect and/or measure at least one neurological and/or cardiopulmonary function of the subject. The headset 11 may also include one or more physiological/environmental sensors, as described above. The electrodes and sensors, and any associated preamp circuitry, if necessary, are collectively illustrated as 76 in FIG. 10. The headset 11 also includes a speaker 72 and microphone 70. The speaker 72 is in electrical communication with the electronic device 14 via an audio output port 14b of the electronic device 14, and the microphone 70 is in electrical communication with the electronic device 14 via an audio input port 14a of the electronic device 14.

Audio information is passed from the electronic device 14 to the headset speaker 72 and audio information from the microphone 70 is transmitted to electronic device 14 via the respective audio input and output ports 14a, 14b. The headset 11 also includes a microcontroller 74 (or a sensor module including a microcontroller or processor) configured to receive and transmit signals produced by the electrodes/sensors 76 to the electronic device for display via a user interface associated with the electronic device 14. The microcontroller 74 is configured to modulate and transmit signals produced by the electrodes/sensors 76 to the electronic device 14 via the audio input port 14b. The sensor data may be modulated by the microcontroller/modulator 74 in such a way that it does not interfere with the audio signal and/or in such a way that it can be easily demodulated by the electronic device 14. Modulation of an electrode signal, such as an ECG signal, can be achieved through an analog modulation technique and/or a digital modulation technique, including, but not limited to amplitude modulation, frequency modulation, phase modulation, phase-shift keying, frequency-shift keying, amplitude-shift keying, quadrature amplitude modulation, continuous phase modulation, wavelet modulation, trellis coded modulation, orthogonal frequency division multiplexing, or the like.

The illustrated embodiment of FIG. 10 is advantageous because it allows the electrodes and sensors to be sampled through the 4-wire audio input/output ports 14a, 14b of the electronic device 14. In addition, it allows multiple sensors to be integrated into the same headset or earbud module with minimal hardware reconfiguration. In some wearable devices, additional input/output ports are not accessible for external hardware not developed by the original manufacturer. In such case, embodiments of the present invention exploit the analog audio input/output ports of the electronic device without disturbing the audio performance of the headset for both audio input (to a headset speaker) and audio output (from a headset microphone).

The microcontroller 74 may digitize both the audio and sensor signals for digital modulation. In another embodiment, this digitally modulated signal may then be converted to an analog modulated signal, preferably an audio modulated signal, via the microcontroller 74 using a digital-to-analog converter (DAC). In this case, an analog signal, as opposed to a digital signal, would pass through the audio input port 14a of the electronic device 14. In other embodiments, the microcontroller 74 may digitize sensor information into a buffer in memory, convert the buffered digital information to an analog signal (via a DAC), and send the analog signal to a modulator for combining the analog microphone audio signal with the analog sensor signal. Converting digital signals back to analog signals may be beneficial because the audio input of the wearable electronic device may not be suited for digital information. The modulator itself may be part of the microcontroller, a separate chip, or a separate circuit.

In some cases, the audio input port 14a of the electronic device 14 may not supply the right level of voltage and/or current. In such case, a power conditioning chip and/or circuit can be implemented to raise or lower the voltage. For example, a voltage multiplier chip may be used to increase the voltage from the audio input port 14a. In some cases, the microcontroller 74 itself may have onboard power conditioning such that additional circuitry is not required.

Although the embodiment of FIG. 10 shows the headset 11 wired to an electronic device 14, it should be understood that wireless versions can also be implemented. The audio input and output lines to and from the headset 11 can be connected to a wireless chip, for generating a wireless signal to be received by a wireless receiver in the wearable electronic device. Examples of wireless chips include, but are not limited to, Bluetooth® chips, ZigBee chips, WiFi chips, and the like. In some cases, the microcontroller 74 itself can be the internal microcontroller of the wireless chip, for a heavily integrated solution. A specific example of this is the Bluecore processor of the Bluecore chip. For even further integration, the entire processing, wireless interface, and modulating electronics can be integrated into an ASIC (application-specific integrated circuit).

In some cases, the analog sensor signals, such as the electrode and/or sensor signals, may pass through the audio input port 14a directly, to be processed further via an embedded computer in the electronic device 14. In such case, the electrode/sensor signals may be processed mostly or entirely by the electronic device 14.

The output of electrodes/sensors 76 can be passed to the electronic device 14 through a wired or wireless configuration. For example, in the wireless configuration, the amplified output from an electrode/sensor 76 can be passed to a wireless processing module, where the wireless processing module can be embedded in the headset 11, as with a Bluetooth® headset. To communicate with the wireless headset 11, the electronic device 14, or associated modules attached to the electronic device 14, are capable of receiving and processing the wireless signal from the wireless headset. Suitable wireless protocols include, but are not limited to, Bluetooth®, ZigBee, WiFi, radio, and several others. In a wired version, the amplified output from an electrode/sensor 76 can be processed in a module embedded in the headset 11, where the resulting signal is passed through one or more wires to the electronic device 14.

In some embodiments of the present invention, an electronic device 14 may contain one or more port(s), capable of wired or wireless contact with a headset 11. These ports are suitable for receiving analog or digitized data from the headset and/or transmitting analog or digitized signals from the electronic device 14 to the headset 11. Examples of such ports include, but or not limited to, Bluetooth® dongles, ZigBee dangles, USB, UART, RS232, Firewire®, optical, proprietary, or other port. In some embodiments, the ports may be connected directly to separate modules that connect in a wired or wireless fashion with a headset 11. These modules may be necessary for conditioning the signals or power levels received by or transmitted to the headset. A Bluetooth®, ZigBee, level translator, mating connector, or DTMF dongle is one example of such a module. These modules may contain signal processing circuitry or components to condition the signals.

As shown in FIG. 10, the signals entering the electronic device 14, sent from the headset 11, may be composed of modulated audio +sensor information. The electronic device 14, serving as an embedded computer, can digitize, demodulate, process, and manipulate this signal internally. The end result is a pure (or mostly pure) audio signal and a separate sensor signal. Through a user interface, such as a graphical user interface (GUI) of the electronic device 14, processed electrode/sensor information can be displayed visually and/or audibly to the user in a colorful and engaging display. The end result is real-time active health and fitness feedback for the headset wearer, while he/she enjoys audio at the same time. In some cases, the feedback may be related through the audio headset itself. ECG signals, EEG signals. EOG signals, core body temperature, physical activity, pulse rate, breathing rate, and other physiological information can be processed by the embedded computer into meaningful assessments such as calories burned, VO₂max, cardiovascular health, and the like.

In some embodiments of the present invention, additional sensors are embedded into the headset 11 for monitoring additional physiological information, noise information (such as motion noise information), and/or environmental exposures of the headset wearer. In such case, an onboard microcontroller 74 (or sensor module comprising a microcontroller or processor) can be used to coordinate the collection, modulation, and transmission of various sensor information. The bi-directional arrow in FIG. 10 between the microcontroller 74 and the electrodes/sensors 76 indicates that bidirectional communication may be employed. In a specific embodiment, the sensors are connected in a serial bus, such as an I2C bus, for poling each sensor and synchronizing the output signal to the wearable electronic device.

The electrodes, as well as additional sensors, can be embedded into a standard audio headset through a variety of processes, including, but not limited to: molding, screen printing, prefabrication, embedded design, encapsulation, or the like. In the specific case of molding, a plastic mold may be generated to fit the desired electrode geometry. As the electrode may be integrated into an electronic module, the mold may be designed to fit the entire module. The module may include all electronic components, including the audio speaker or audio microphone. Screen printing conductive electrodes can be useful for printing over existing, prefabricated headsets. In some cases, the metal enclosures from the headsets or headset speakers themselves can serve as an electrode. In the case of wired headsets, additional wires may be added to connect with ports in the wearable electronic device.

The electrodes described herein can also be used to measure the EEG and/or EOG of a person wearing the headset. Extracting EEG and EOG signals in the midst of ECG signals can be achieved using several methods. One method is to place the electrodes in locations closest to a region of interest. For example, integrating EOG sensors in a headset fixture close to the eyes would improve the response to the EOG. Another method is to integrate multiple electrodes at various regions on a single earpiece. As a specific example, having two separate electrodes in each earpiece of a stereo headset would provide a way of differentiating EOG, EEG, and ECG signals. This is because the localized potential between the two closely space electrodes in a single earbud can be more indicative of localized EOG and EEG events, whereas the more distal potential between electrodes in separate earbuds can be more indicative the ECG response.

Although FIG. 10 illustrates the headset 11 wired to an electronic device 14, it should be understood that wireless versions can also be implemented, according to some embodiments of the present invention. For example, the audio input and output lines to and from the headset 11 can be connected to a wireless chip, for generating a wireless signal to be received by a wireless receiver in the electronic device 14. Examples of wireless chips include, but are not limited to, Bluetooth® chips, ZigBee chips, WiFi chips, and the like. In some embodiments, the microcontroller 74 itself can be the internal microcontroller of the wireless chip, for a heavily integrated solution. A specific example of this is the Bluecore processor of the Bluecore chip. For even further integration, the entire processing, wireless interface, and modulating electronics can be integrated into an ASIC (application-specific integrated circuit).The microcontroller 74 in the illustrated embodiment of FIG. 12 may integrate the sensor, processor, and wireless electronics to communicate with a remote device. In this way, the phone jack may power the microcontroller and the microcontroller may wirelessly communicate with a remote device.

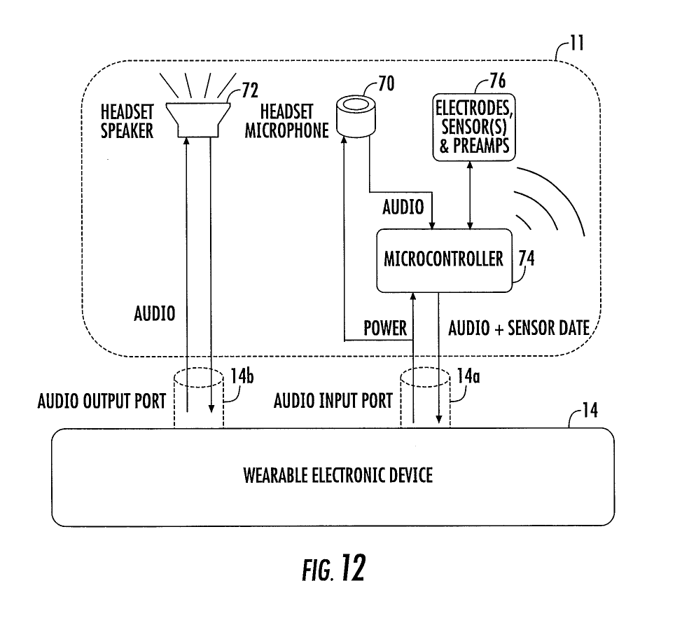

FIG. 11 is a block diagram illustrating that circuitry for sensing and processing electrical signals from the body of a subject may be integrated into a sensor module. For example, the sensor module represented by FIG. 11 may replace the microcontroller 74 illustrated in FIGS. 10 and 12, according to some embodiments of the present invention. As illustrated in FIG. 11, a sensor module, according to embodiments of the present invention, may include circuitry for power conditioning, signal conditioning, A/D and D/A conversion, wireless transmission, controls, and the like. For example, in some embodiments, the sensor module may comprise a microcontroller, sensor, and a wireless transmitter.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A monitoring apparatus, comprising:
a housing configured to be attached to an ear of a subject; and
a plurality of electrodes supported by the housing, wherein the electrodes are configured to at least partially contact a portion of the body of the subject when the housing is attached to the ear of the subject, and wherein the electrodes are configured to detect and/or measure at least one neurological and/or cardiopulmonary function of the subject.

2. The apparatus of claim 1, wherein the electrodes are electrocardiogram (ECG) electrodes, electroencephalogram (EEG) electrodes, and/or electrooculography (EOG) electrodes.

3. The apparatus of claim 1, wherein the electrodes are spaced-apart from each other and are electrically connected via a circuit board.

4. The apparatus of claim 3, wherein the circuit board is a flexible circuit board.

5. The apparatus of claim 1, further comprising a physiological sensor supported by the housing and configured to detect and/or measure physiological information from the subject.

6. The apparatus of claim 1, further comprising an environmental sensor supported by the housing and configured to detect and/or measure environmental conditions in a vicinity of the subject.

7. The apparatus of claim 1, further comprising one or more biasing members configured to urge the electrodes into contact with the body of the subject when the housing is attached to the ear of the subject.

8. The apparatus of claim 1, further comprising an ear clip attached to the housing that facilitates attachment of the housing to the ear of a subject, wherein the ear clip includes one or more electrodes configured to at least partially contact a portion of the body of a subject when the housing is attached to the ear of the subject.

9. The apparatus of claim 8, wherein the ear clip comprises a pinna cover, and wherein the pinna cover includes one or more electrodes configured to at least partially contact a portion of an ear of a subject when the housing is attached to the ear of the subject.

10. The apparatus of claim 1, wherein the housing comprises an earbud configured to be inserted within an ear of a subject and wherein the electrodes are supported by the earbud.

11. The apparatus of claim 1, further comprising a sensor module supported by the housing and configured to amplify and/or filter signals produced by the electrodes.

12. The apparatus of claim 11, further comprising a transmitter supported by the housing and configured to transmit signals processed by the sensor module to a remote device.

13. The apparatus of claim 11, further comprising a speaker and microphone supported by the housing, wherein the speaker is configured to be in electrical communication with an electronic device via an audio output port of the electronic device, wherein the microphone is configured to be in electrical communication with the electronic device via an audio input port of the electronic device, and wherein the sensor module is configured to modulate and transmit signals produced by the electrodes to the electronic device via the audio input port.

14. The apparatus of claim 11, wherein the sensor module is configured to wirelessly transmit signals produced by the electrodes to a remote electronic device.

15. The apparatus of claim 13, wherein the sensor module is configured to digitize signals produced by the electrodes.

16. The apparatus of claim 13, further comprising a power conditioning component supported by the housing and configured to adjust voltage and/or current to the sensor module.

17. A monitoring apparatus, comprising:
a headset configured to be worn by a subject; and
an electronic device comprising a user interface;
wherein the headset comprises a plurality of electrodes configured to at least partially contact a portion of the body of the subject when the headset is worn by the subject, and wherein the electrodes are configured to detect and/or measure at least one neurological and/or cardiopulmonary function of the subject;
wherein the headset comprises a sensor module configured to receive and transmit signals produced by the electrodes to the electronic device for display via the user interface.

18. The apparatus of claim 17, wherein the electrodes are electrocardiogram (ECG) electrodes, electroencephalogram (EEG) electrodes, and/or electrooculography (EOG) electrodes.

19. The apparatus of claim 17, wherein the electrodes are spaced-apart from each other and are electrically connected via a circuit board.

20. The apparatus of claim 19, wherein the circuit board is a flexible circuit board.

21. The apparatus of claim 17, wherein the headset comprises one or more physiological sensors configured to detect and/or measure physiological information from the subject.

22. The apparatus of claim 17, wherein the headset comprises one or more environmental sensors configured to detect and/or measure environmental conditions in a vicinity of the subject.

23. The apparatus of claim 17, wherein the headset comprises one or more biasing members configured to urge the electrodes into contact with the body of the subject when the headset is worn by the subject.

24. The apparatus of claim 17, wherein the sensor module is configured to amplify and/or filter signals produced by the electrodes.

25. The apparatus of claim 17, wherein the headset comprises a speaker and a microphone, wherein the speaker is in audio communication with the electronic device via an audio output port of the electronic device, wherein the microphone is in audio communication with the electronic device via an audio input port of the electronic device, and wherein the sensor module is configured to modulate and transmit signals produced by the electrodes to the electronic device via the audio input port.

26. The apparatus of claim 17, wherein the electronic device is configured to be worn by the subject.

27. The apparatus of claim 17, wherein the headset comprises two earbuds connected by a supporting member, each earbud configured to be inserted within an ear of a subject, wherein one or more of the electrodes are supported by the supporting member and/or wherein one or more of the electrodes are supported by at least one earbud.

28. The headset of claim 27, wherein the supporting member comprises one or more biasing members configured to urge the one or more electrodes into contact with the body of the subject when the headset is worn by the subject.

29. The headset of claim 27, wherein the at least one earbud comprises one or more biasing members configured to urge the one or more electrodes into contact with the ear of the subject when the headset is worn by the subject.

30. A headset, comprising:
two earbuds connected by a supporting member, each earbud configured to be inserted within an ear of a subject;
one or more electrodes supported by the supporting member and configured to at least partially contact a portion of the body of the subject when the headset is worn by the subject; and
one or more electrodes supported by each earbud and configured to at least partially contact a portion of an ear of the subject when the headset is worn by the subject;
wherein the electrodes are configured to detect and/or measure at least one neurological and/or cardiopulmonary function of the subject.

31. The headset of claim 30, wherein the electrodes are electrocardiogram (ECG) electrodes, electroencephalogram (EEG) electrodes, and/or electrooculography (EOG) electrodes.

32. The headset of claim 30, further comprising at least one physiological sensor configured to detect and/or measure physiological information from the subject.

33. The headset of claim 30, further comprising at least one environmental sensor configured to detect and/or measure environmental conditions in a vicinity of the subject.

34. The headset of claim 30, wherein the supporting member comprises one or more biasing members configured to urge the one or more electrodes into contact with the body of the subject when the headset is worn by the subject.

35. The headset of claim 30, wherein each earbud comprises one or more biasing members configured to urge the one or more electrodes into contact with the ear of the subject when the headset is worn by the subject.

36. The headset of claim 30, further comprising a sensor module configured to amplify and/or filter signals produced by the electrodes.

37. The headset of claim 36, further comprising a transmitter configured to transmit signals processed by the sensor module to a remote device.

38. A monitoring apparatus, comprising:
a housing configured to be attached to an ear of a subject;
a first electrode supported by the housing and configured to at least partially contact a portion of the body of the subject when the housing is attached to the ear of the subject;
an earring configured to be attached to the ear of the subject; and
a second electrode supported by the earring and configured to at least partially contact a portion of the ear of the subject when the earring is attached to the ear of the subject;
wherein the first and second electrodes are configured to detect and/or measure at least one neurological and/or cardiopulmonary function of the subject.

39. The apparatus of claim 38, wherein the first and second electrodes are electrocardiogram (ECG) electrodes, electroencephalogram (EEG) electrodes, and/or electrooculography (EOG) electrodes.

40. The apparatus of claim 38, further comprising a sensor module supported by the housing and configured to amplify and/or filter signals produced by the first and second electrodes.

41. The apparatus of claim 40, further comprising a transmitter supported by the housing and configured to transmit signals processed by the sensor module to a remote device.

42. A method of monitoring a subject, comprising:
detecting neurological and/or cardiopulmonary function information from the subject via electrodes attached to a headset worn by the subject;
transmitting the information to a remote electronic device via an audio input port of the remote electronic device.

43. The method of claim 42, wherein the electrodes are electrocardiogram (ECG) electrodes, electroencephalogram (EEG) electrodes, and/or electrooculography (EOG) electrodes.

44. The method of claim 42, wherein the headset comprises a speaker and a microphone, wherein the speaker is in audio communication with the electronic device via an audio output port of the electronic device, wherein the microphone is in audio communication with the electronic device via an audio input port of the electronic device, and wherein transmitting the information to the remote electronic device comprises modulating the information and transmitting the information with audio signals produced by the microphone.
